(19) United States
(12) Patent Application Publication
(10) Pub. No.: US 2022/0008578 A1
(43) Pub. Date: Jan. 13, 2022

(54) PACKAGE RECEIVING LOCKER

(71) Applicant: Securum Capsa, Inc., Los Angeles, CA (US)

(72) Inventors: Carlos Raphael, Los Angeles, CA (US); Jeff Harlan, Corona, CA (US)

(73) Assignee: Securum Capsa, Inc., Los Angeles, CA (US)

(21) Appl. No.: 17/327,544

(22) Filed: May 21, 2021

Related U.S. Application Data
(63) Continuation-in-part of application No. 16/920,615, filed on Jul. 3, 2020.
Application No. 16/920,615 is a continuation-in-part of application No. 17/086,389, filed on Oct. 31, 2020.
(60) Provisional application No. 63/028,330, filed on May 21, 2020.

(51) Int. Cl. A61L 2/10; B65D 6/18; E05B 65/02

(52) U.S. Cl. CPC A61L 2/10; B65D 9/14; A61L 2202/122; A61L 2202/23; A61L 2202/11; E05B 65/025

(57) ABSTRACT

A collapsible-locker, that is lockable, for receiving packages, may have at least one sterilizer for sterilizing at least some exterior portion of a given package received into the collapsible-locker. The collapsible-locker may have a sterilizer for sterilizing at least some exterior portions of the collapsible-locker where a person may be intended to interact with the collapsible-locker. The collapsible-locker may have a lid, a front-wall, a rear-wall, side-walls, and a floor that form main aspects of the collapsible-locker. Interior surfaces of the lid, the front-wall, the rear-wall, the side-walls, and/or the floor may be reflective to enhance UV (ultraviolet) light sterilizing the at least some exterior portion of the given package received into the collapsible-locker. The collapsible-locker may be configured to be attached to a side hinged door, wherein the hinged door provides access to a building for people.

### PRIORITY NOTICE

The present application claims priority under 35 U.S.C. § 119(e) to U.S. Provisional Patent Application Ser. No. 63/028,330 filed on May 21, 2020, the disclosure of which is incorporated herein by reference in its entirety.

The present application claims priority under 35 U.S.C. § 120 to U.S. Nonprovisional patent application Ser. No. 16/920,615 filed on Jul. 3, 2020, the disclosure of which is incorporated herein by reference in its entirety. The present application is a continuation-in-part (CIP) of U.S. Nonprovisional patent application Ser. No. 16/920,615 filed on Jul. 3, 2020.

The present application claims priority under 35 U.S.C. § 120 to U.S. Nonprovisional patent application Ser. No. 17/086,389 filed on Oct. 31, 2020, the disclosure of which is incorporated herein by reference in its entirety. The present application is a continuation-in-part (CIP) of U.S. Nonprovisional patent application Ser. No. 17/086,389 filed on Oct. 31, 2020.

### TECHNICAL FIELD OF THE INVENTION

The present invention relates in general to package receiving lockers and more specifically to package receiving lockers with various sterilizing capabilities.

### COPYRIGHT AND TRADEMARK NOTICE

A portion of the disclosure of this patent application may contain material that is subject to copyright protection. The owner has no objection to the facsimile reproduction by anyone of the patent document or the patent disclosure, as it appears in the Patent and Trademark Office patent file or records, but otherwise reserves all copyrights whatsoever.

Certain marks referenced herein may be common law or registered trademarks of third parties affiliated or unaffiliated with the applicant or the assignee. Use of these marks is by way of example and should not be construed as descriptive or to limit the scope of this invention to material associated only with such marks.

### BACKGROUND OF THE INVENTION

The 2019-2020 corona virus/SARS-CoV-2 virus/COVID-19 pandemic resulted in an increase of products being directly delivered to residences and businesses, including that of food and beverage goods, as many sought to minimize their exposure to the virus and/or as former retail outfits were required to close or enact various protocols to minimize exposure to the virus.

However, with this increase in the direct delivery of products to residences and/or businesses, a need to clean, sanitize, and/or sterilize those delivered products has also grown—as such delivered products may be non-sterile and may be harboring dangerous pathogens.

But this pandemic has resulted in shortages of various mostly liquid based cleaners, sanitizers, and/or sterilizers, including but not limited to bleach, alcohol (rubbing/isopropyl), dish soaps, hand soaps, and the like. Regardless of such shortages, some materials (e.g., paper and/or cardboard based boxes, envelopes, and/or bags) are not amendable to being treated with liquids, as they will and can deteriorate when wet. Also, some liquid treatment products, like bleach, may be too harsh for some packaging materials.

There is a need in the art for a means to at least sterilize exterior surfaces of such delivered products and to do so without harming/destroying the integrity of the given shipping article (e.g., envelope, box, and/or bag).

In addition, to the problem of sterilizing at least some exterior surfaces of such delivered products, there is also the problem that delivered products are often stolen, i.e., the problem of porch piracy.

It is to these ends that the present invention has been developed.

### BRIEF SUMMARY OF THE INVENTION

To minimize the limitations in the prior art, and to minimize other limitations that will be apparent upon reading and understanding the present specification, the present invention may describe collapsible-lockers, with locking lids, that may be removably attachable to a given side hinged door, wherein such a side hinged door is the common type of door that provides human access to a given building (e.g., an exterior door of a residence and/or building with at least one hinge along one side). Such collapsible-lockers address the problems of porch piracy.

In some embodiments, such collapsible-lockers may also address the problem of sterilizing at least some exterior surfaces of delivered products. In some embodiments, a given collapsible-locker may comprise at least one sterilizer for sterilizing at least some exterior portion of a given package received into the collapsible-locker. In some embodiments, the collapsible-locker may comprise a sterilizer for sterilizing at least some exterior portions of the collapsible-locker where a person may be intended to interact with the collapsible-locker. In some embodiments, the collapsible-locker may comprise a lid, a front-wall, a rear-wall, side-walls, and a floor. In some embodiments, at least some interior surfaces of the lid, the front-wall, the rear-wall, the side-walls, and/or the floor may be reflective to enhance UV (ultraviolet) light sterilizing the at least some exterior portion of the given package received into the collapsible-locker. In some embodiments, the collapsible-locker may be configured to be attached to a side hinged door, wherein the hinged door provide access to a building for people.

It is an objective of the present invention to provide a collapsible-locker with at least one sterilizer.

It is another objective of the present invention to provide a collapsible-locker with at least one (internal) sterilizer oriented to sterilize at least some exterior portions of a package received into the collapsible-locker.

It is another objective of the present invention to provide a collapsible-locker with at least some interior surfaces being highly reflective to enhance UV (ultraviolet) light sterilization that may be emitted by the at least one internal sterilizer.

It is yet another objective of the present invention to provide a collapsible-locker with at least one (external) sterilizer oriented to sterilize at least some exterior portions of the collapsible-locker where a person might be directed to engage with the given collapsible-locker, so as to provide a sterile exterior surface region of the collapsible-locker where the person may interact/engage with.

These and other advantages and features of the present invention are described herein with specificity so as to make the present invention understandable to one of ordinary skill in the art, both with respect to how to practice the present invention and how to make the present invention.

### BRIEF DESCRIPTION OF THE SEVERAL VIEWS OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention.

FIG. 1 shows a left, front, and top perspective view of a collapsible-locker in its fully expanded configuration with its lid at least partially open so that some interior portions of the collapsible-locker may be seen.

FIG. 2 shows a front view of the collapsible-locker of FIG. 1 and in the same configuration as shown in FIG. 1.

FIG. 3 shows a left, front, and top perspective view of the collapsible-locker of FIG. 1, in its fully expanded configuration, but now with its lid closed.

FIG. 4 shows a front view of the collapsible-locker of FIG. 3 and in the same configuration (e.g., with its lid closed) as shown in FIG. 3.

### REFERENCE NUMERAL SCHEDULE

101 collapsible-locker 101
103 front-wall 103
105 side-wall 105
107 side-wall-panel-closer-to-rear-wall 107
109 side-wall-panel-closer-to-front-wall 109
111 lid 111
113 lid-overhang 113
115 exterior-interface 115
119 rear-wall 119
171 sterilizer 171
173 reflective-surface 173
175 sterilizer 175
181 latch lock 181
183 latch lock receiver 183
185 latch lock receiver 185
191 light/sensor 191 (or screw hole 191)
193 light/sensor 193
195 light/sensor 195 (or injection molding artefact 195)
199 volume 199
351 pod-receiver 351
2905 interior-camera 2905

### DETAILED DESCRIPTION OF THE INVENTION

Component/item/element/part/structural names used herein, typically associated with a given reference numeral, are intended to imply and/or suggest a structure and/or function by that name. For example, a name of “collapsible-locker” is intended to be a name for a device/apparatus, that functions as a locker, but that is collapsible.

In the following discussion that addresses a number of embodiments and applications of the present invention, reference is made to the accompanying drawings that form a part thereof, where depictions are made, by way of illustration, of specific embodiments in which the invention may be practiced. It is to be understood that other embodiments may be utilized and changes may be made without departing from the scope of the invention.

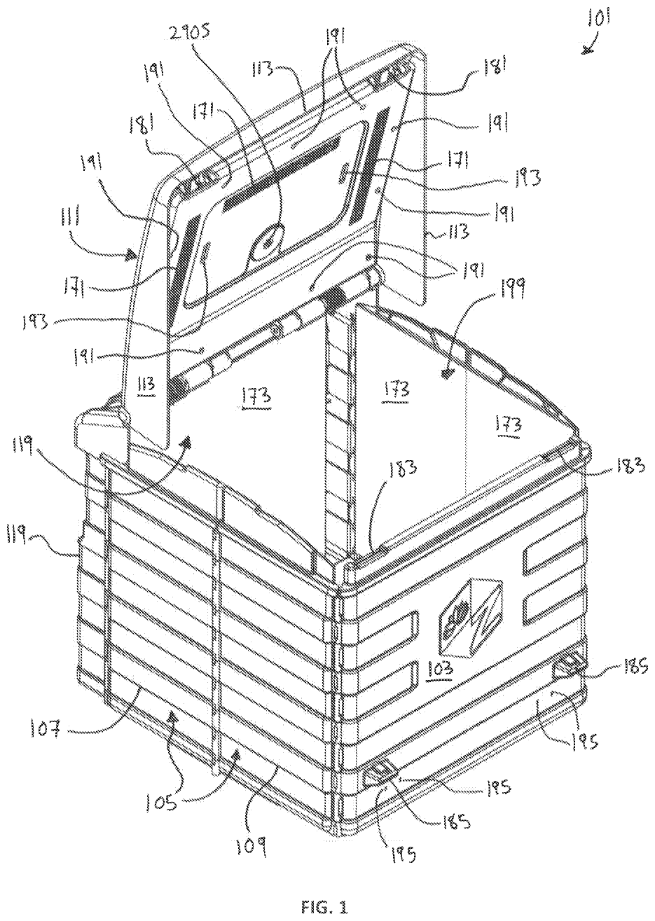

FIG. 1 shows a left, front, and top perspective view of a collapsible-locker 101 in its fully expanded configuration with its lid 111 at least partially open so that some interior portions of collapsible-locker 101 may be seen. For example, and without limiting the scope of the present invention, some underside portions of lid 111 may be seen in FIG. 1. For example, and without limiting the scope of the present invention, some interior portions of side-wall 105 surfaces of collapsible-locker 101 may be seen in FIG. 1. For example, and without limiting the scope of the present invention, some interior portions of a rear-wall 119 surfaces of collapsible-locker 101 may be seen in FIG. 1. For example, and without limiting the scope of the present invention, some portions of volume 199 of collapsible-locker 101 may be seen in FIG. 1.

Continuing discussing FIG. 1, in some embodiments, collapsible-locker 101 may be substantially as shown and described as the various collapsible-lockers (e.g., collapsible-lockers 101) shown and described in U.S. nonprovisional patent application Ser. No. 16/414,634, filed on May 16, 2019, the disclosure of which is incorporated herein by reference in its entirety. In some embodiments, collapsible-locker 101 may be substantially as shown and described as the various collapsible-lockers (e.g., collapsible-lockers 101) shown and described in U.S. provisional patent application Ser. No. 62/929,201 filed on Nov. 1, 2019, the disclosure of which is incorporated herein by reference in its entirety. In some embodiments, collapsible-locker 101 as shown and described herein (in this patent application) may differ only in that this collapsible-locker 101 may also comprise at least one sterilizer 171/175 and/or may comprise interior reflective surfaces 173.

Continuing discussing FIG. 1, in some embodiments, collapsible-locker 101 may be exist in two main operational configurations, a substantially expanded (unfolded) configuration, as shown herein; and in a substantially collapsed (folded) configuration not shown herein, but that is shown in U.S. nonprovisional patent application Ser. No. 16/414,634 and in U.S. provisional patent application Ser. No. 62/929,201.

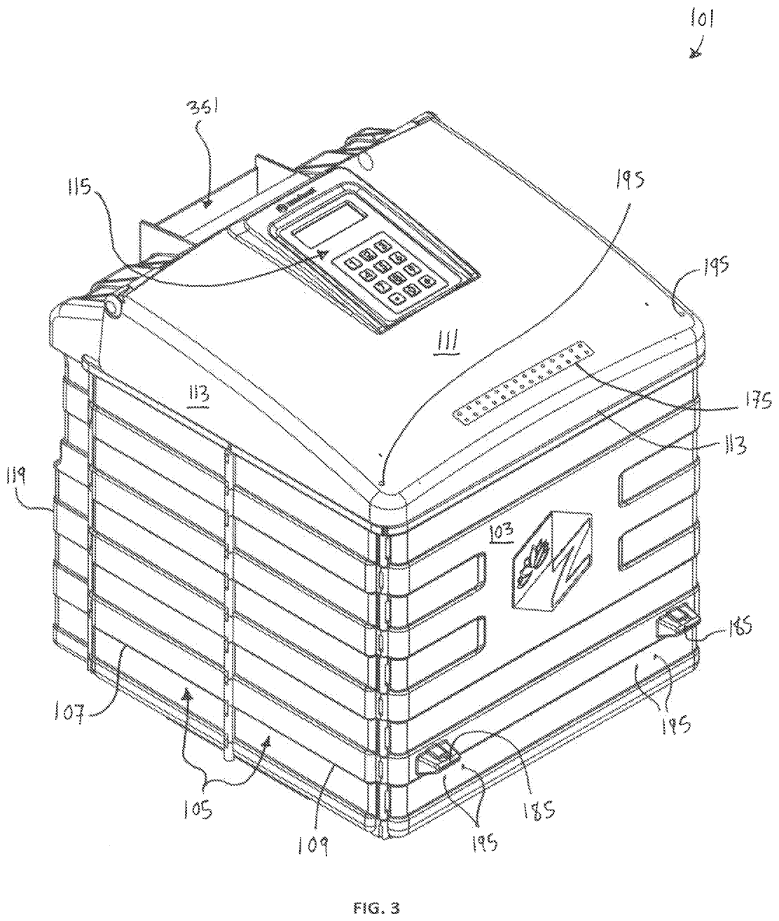

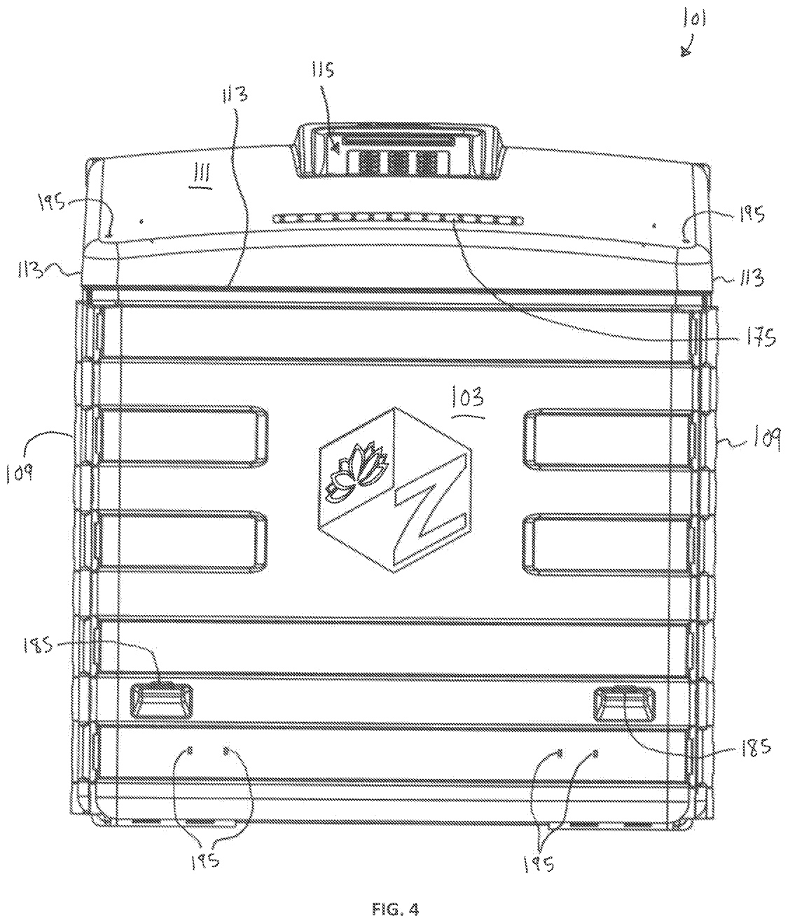

Continuing discussing FIG. 1, in some embodiments, when collapsible-locker 101 may be in its substantially expanded (unfolded) configuration (shown herein) collapsible-locker 101 may be a locker, a box, a container, and/or the like that substantially encloses a hollow volume 199. In some embodiments, this hollow volume 199 may be intended for the temporary receival and/or storage of various packages, parcels, boxes, containers, envelopes, bags, shipping bags, combinations thereof, and/or the like. In some embodiments, volume 199 may be substantially and/or entirely bound (enclosed) by various structural elements of collapsible-locker 101, such as, but not limited to, a front-wall 103, side-walls 105, rear-wall 119, lid 111, and a floor 121 (not shown herein). In some embodiments, front-wall 103 and rear-wall 119 may be substantially parallel with each other and disposed opposite from each other. In some embodiments, a set of two side-walls 105 may be substantially parallel with each other and disposed opposite from each other. In some embodiments, portions of lid 111 and floor 121 may be substantially parallel with each other and disposed opposite from each other, when lid 111 may be closed (see e.g., FIG. 3 and FIG. 4 for when lid 111 is closed). In some embodiments, front-wall 103 may be hingedly attached to the set of side-walls 105. In some embodiments, rear-wall 119 may be hingedly attached to the set of side-walls 105, to lid 111, and to floor 121. In some embodiments, each side-wall 105 may be also be folded in substantially half, with respect to a vertical direction from floor 121 to lid 111. In some embodiments, each side-wall 105 may be comprised of two smaller side-wall portions, side-wall-panel-closer-to-rear-wall 107 and side-wall-panel-closer-to-front-wall 109, respectively. In some embodiments, side-wall-panel-closer-to-rear-wall 107 may be hingedly attached to side-wall-panel-closer-to-front-wall 109. In some embodiments, side-wall-panel-closer-to-rear-wall 107 may also be hingedly attached to rear-wall 119. In some embodiments, side-wall-panel-closer-to-front-wall 109 may also be hingedly attached to front-wall 103.

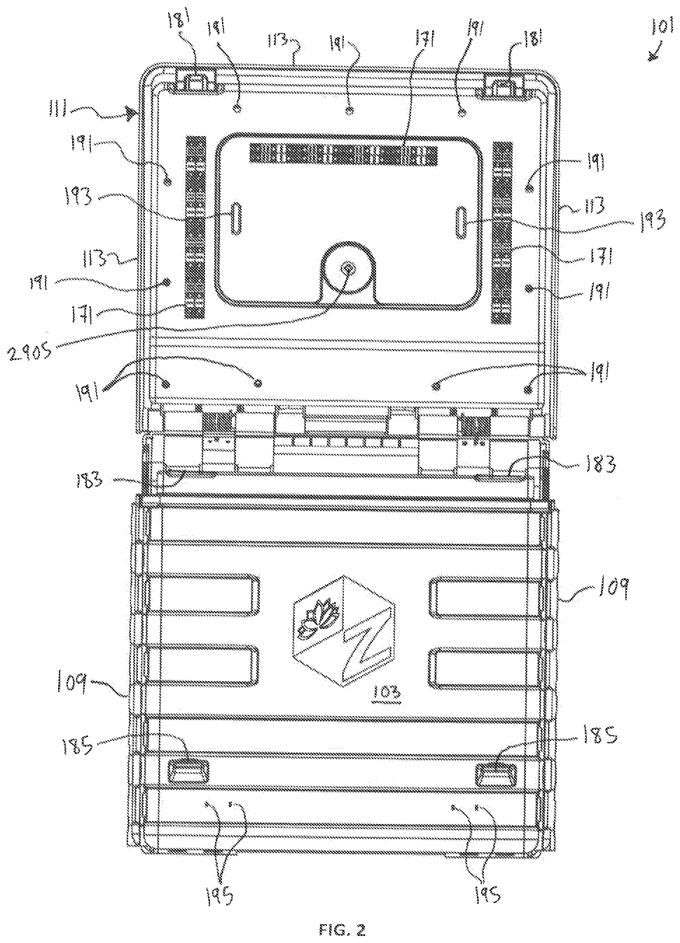

Continuing discussing FIG. 1, in some embodiments, lid 111 may comprise at least one sterilizer 171 and/or sterilizer 175. Note, sterilizer 175 may be shown in FIG. 3 and in FIG. 4; whereas sterilizer 171 may be shown in FIG. 1 and in FIG. 2. Continuing discussing FIG. 1, in some embodiments, the at least one sterilizer 171 may be located on an interior surface of collapsible-locker 101, that emits at least some electromagnetic energy into and/or towards volume 199. In some embodiments, an interior surface of collapsible-locker 101 may comprise at least one opening configured to emit at least some electromagnetic energy from at least one sterilizer 171.

Continuing discussing FIG. 1, in some embodiments, an underside of lid 111 may comprise at least one sterilizer 171. In some embodiments, an interior surface of lid 111 may comprise at least one sterilizer 171. In some embodiments, an underside of lid 111 may comprise at least one opening configured to emit at least some electromagnetic energy from at least one sterilizer 171. In some embodiments, an interior surface of lid 111 may comprise at least one opening configured to emit at least some electromagnetic energy from at least one sterilizer 171.

In some embodiments, the at least one sterilizer 171 may be attached to: lid 111, front-wall 103, side-wall 105, side-wall-panel-closer-to-rear-wall 107, side-wall-panel-closer-to-front-wall 109, rear-wall 119, floor 121, combinations thereof, and/or the like.

In some embodiments, the at least one sterilizer 171 and/or the at least one sterilizer 175 may be operatively connected to (e.g., wired) to various electronics of collapsible-locker 101, such as, but not limited to, Processors 1501, I/O Means 1507, power-supply 1509, batteries, battery pack, bus, switches, circuits, combinations thereof, and/or the like.

Continuing discussing FIG. 1, in some embodiments, sterilizer 171 and/or sterilizer 175 may be configured to emit electromagnetic energy of a predetermined characteristic (e.g., a predetermined wavelength or ranges of wavelengths). In some embodiments, sterilizer 171 and/or sterilizer 175 may be configured to emit ionizing radiation. In some embodiments, sterilizer 171 and/or sterilizer 175 may be configured to emit electromagnetic energy configured to destroy genetic material. In some embodiments, sterilizer 171 and/or sterilizer 175 may be configured to emit electromagnetic energy configured to denature genetic material. In some embodiments, sterilizer 171 and/or sterilizer 175 may be configured to emit electromagnetic energy configured to denature DNA (deoxynucleic acid) and/or RNA (ribonucleic acid). In some embodiments, sterilizer 171 and/or sterilizer 175 may be configured to emit electromagnetic energy configured to kill single cell organisms, such as, bacteria, fungi, algae, and/or protozoa. In some embodiments, sterilizer 171 and/or sterilizer 175 may be configured to emit electromagnetic energy configured to render a virus incapable of infection. In some embodiments, sterilizer 171 and/or sterilizer 175 may be configured to emit electromagnetic energy configured to render a virus incapable of reproducing. In some embodiments, electromagnetic energy emitted from sterilizer 171 and/or from sterilizer 175 may be one or more of antiviral, antibacterial, antifungal, antimicrobial, combinations thereof, and/or the like. In some embodiments, sterilizer 171 and/or sterilizer 175 may be a light source. In some embodiments, sterilizer 171 and/or sterilizer 175 may be a UV (ultraviolet) light source. In some embodiments, sterilizer 171 and/or sterilizer 175 may be configured to emit substantially UV spectrum often referred to as UVC. In some embodiments, sterilizer 171 and/or sterilizer 175 may be one or more of a light emitting diode (LED) (in strip format in some embodiments), incandescent light bulb, fluorescent light tube/bulb, cathode tube/bulb, a transducer, an antenna, a frequency generator, a frequency modulator, a lens, a reflector, a refractor, a transformer, a circuit, a switch, combinations thereof, and/or the like, configured to emit electromagnetic energy/radiation of a predetermined wavelength, such as, but not limited to, UV light of a predetermined spectrum (such as, but not limited to, UVC). In some embodiments, sterilizer 171 and/or sterilizer 175 may comprise one or more of a light emitting diode (LED) (in strip format in some embodiments), incandescent light bulb, fluorescent light tube/bulb, cathode tube/bulb, a transducer, an antenna, a frequency generator, a frequency modulator, a lens, a reflector, a refractor, a transformer, a circuit, a switch, combinations thereof, and/or the like, configured to emit electromagnetic energy/radiation of a predetermined wavelength, such as, but not limited to, UV light of a predetermined spectrum (such as, but not limited to, UVC). In some embodiments, sterilizer 171 and/or sterilizer 175 may comprise other electronics for their function. In some embodiments, sterilizer 171 and/or sterilizer 175 may be well understood and well characterized by the sterilizing arts, wherein such understandings and characterizations are incorporated by reference. In some embodiments, sterilizer 171 and/or sterilizer 175 may be off the shelf components.

Continuing discussing FIG. 1, in some embodiments, at least some interior surfaces of collapsible-locker 101 may be reflective-surface 173. In some embodiments, most (a majority) interior surfaces of collapsible-locker 101 may be reflective-surface 173. In some embodiments, about half of interior surfaces of collapsible-locker 101 may be reflective-surface 173. In some embodiments, at least half of interior surfaces of collapsible-locker 101 may be reflective-surface 173. In some embodiments, at least a portion of: lid 111, front-wall 103, side-wall 105, side-wall-panel-closer-to-rear-wall 107, side-wall-panel-closer-to-front-wall 109, rear-wall 119, floor 121, combinations thereof, and/or the like, may comprise reflective-surface 173. In some embodiments, reflective-surface 173 may be configured to reflect at least some of the electromagnetic energy emitted from sterilizer(s) 171. In some embodiments, reflective-surface 173 may be configured to enhance efficacy of sterilizer(s) 171. In some embodiments, reflective-surface 173 may be configured to reflect at least some UV light. In some embodiments, reflective-surface 173 may be substantially polished. In some embodiments, reflective-surface 173 may be substantially polished. In some embodiments, reflective-surface 173 may be mirror like. In some embodiments, reflective-surface 173 may be an interior surface reflective: film, coating, layer, sheet, deposit, laminate, paint, foil, combinations thereof, and/or the like.

Continuing discussing FIG. 1, in some embodiments, collapsible-locker 101 may comprise at least one interior-camera 2905. In some embodiments, interior-camera 2905 may be configured for monitoring volume 199 and/or the contents of volume 199. In some embodiments, interior-camera 2905 may be substantially as shown and/or described in U.S. nonprovisional patent application Ser. No. 16/414,634, and/or in U.S. provisional patent application Ser. No. 62/929,201.

Continuing discussing FIG. 1, in some embodiments, interior-camera 2905 may be located on an interior surface of collapsible-locker 101, so that interior-camera 2905 may be oriented to monitor volume 199 and/or contents of volume 199. In some embodiments, an interior surface of collapsible-locker 101 may comprise at least one opening configured to provide a lens of interior-camera 2905 access for monitoring volume 199 and/or the contents of volume 199.

Continuing discussing FIG. 1, in some embodiments, an underside of lid 111 may comprise at least one interior-camera 2905. In some embodiments, an interior surface of lid 111 may comprise at least one interior-camera 2905. In some embodiments, an underside of lid 111 may comprise at least one opening for providing a lens of interior-camera 2905 access for monitoring volume 199 and/or the contents of volume 199. In some embodiments, an interior surface of lid 111 may comprise at least one opening for providing a lens of interior-camera 2905 access for monitoring volume 199 and/or the contents of volume 199.

In some embodiments, the at least one interior-camera 2905 may be attached to: lid 111, front-wall 103, side-wall 105, side-wall-panel-closer-to-rear-wall 107, side-wall-panel-closer-to-front-wall 109, rear-wall 119, floor 121, combinations thereof, and/or the like.

In some embodiments, the at least one interior-camera 2905 may be operatively connected to (e.g., wired) to various electronics of collapsible-locker 101, such as, but not limited to, Processors 1501, I/O Means 1507, power-supply 1509, batteries, battery pack, bus, switches, circuits, combinations thereof, and/or the like.

Continuing discussing FIG. 1, in some embodiments, collapsible-locker 101 may comprise at least one light/sensor 191/193. In some embodiments, reference numeral 191 may refer to a light, a light source, a sensor, a hole, a screw hole, combinations thereof, and/or the like. In some embodiments, reference numeral 193 may refer to a light, a light source, a sensor, combinations thereof, and/or the like. In some embodiments, light/sensor 191/193 may be a member of I/O Means 1507. In some embodiments, light/sensor 191 and/or light/sensor 193 may be one or more of: a light source, a light bulb, a light tube, a light emitting diode (LED) (in strip format in some embodiments), incandescent light bulb, fluorescent light tube/bulb, cathode tube/bulb, combinations thereof, and/or the like. In some embodiments, light/sensor 191 and/or light/sensor 193 may be configured and/or oriented to emit at least some visual light into volume 199, when lid 111 may be at least partially open, for a purpose of assisting a person with seeing the contents of volume 199. In some embodiments, light/sensor 191 and/or light/sensor 193 may be configured and/or oriented to emit at least some visual light for a purpose of indicating status, warning, alarms, combinations thereof, and/or the like.

Continuing discussing FIG. 1, in some embodiments, light/sensor 191 and/or light/sensor 193 may be one or more of: a sensor, a thermometer, a thermocouple, a temperature probe, a light meter, a motion detector, radar, sonar, a laser, an accelerometer, a hygrometer, a moisture detector, a water detector, a smoke detector, a particulate detector, combinations thereof, and/or the like. In some embodiments, light/sensor 191 and/or light/sensor 193 may be configured and/or oriented to sense and/or detect at least one condition of volume 199 and/or at least one condition of the contents of volume 199.

Continuing discussing FIG. 1, in some embodiments, light/sensor 191 and/or light/sensor 193 may be located on an interior surface of collapsible-locker 101. In some embodiments, an interior surface of collapsible-locker 101 may comprise at least one opening configured to provide light/sensor 191 and/or light/sensor 193 access for lighting and/or sensing a condition of volume 199 and/or of the contents of volume 199.

Continuing discussing FIG. 1, in some embodiments, an underside of lid 111 may comprise at least one light/sensor 191 and/or light/sensor 193. In some embodiments, an interior surface of lid 111 may comprise at least one light/sensor 191 and/or light/sensor 193. In some embodiments, an underside of lid 111 may comprise at least one opening for light/sensor 191 and/or light/sensor 193 to access volume 199 and/or the contents of volume 199. In some embodiments, an interior surface of lid 111 may comprise at least one opening for light/sensor 191 and/or light/sensor 193 to access for volume 199 and/or the contents of volume 199.

In some embodiments, the at least one light/sensor 191 and/or light/sensor 193 may be attached to: lid 111, front-wall 103, side-wall 105, side-wall-panel-closer-to-rear-wall 107, side-wall-panel-closer-to-front-wall 109, rear-wall 119, floor 121, combinations thereof, and/or the like.

In some embodiments, a plurality of lights/sensors 191 may be operatively connected to themselves. In some embodiments, a plurality of lights/sensors 193 may be operatively connected to themselves. In some embodiments, the at least one light/sensor 191 may be operatively connected to light/sensor 193. In some embodiments, the at least one light/sensor 191 and/or light/sensor 193 may be operatively connected to (e.g., wired) to various electronics of collapsible-locker 101, such as, but not limited to, Processors 1501, I/O Means 1507, power-supply 1509, batteries, battery pack, bus, switches, circuits, combinations thereof, and/or the like.

Note in some embodiments, at least one of the reference numerals 191 may instead refer to a screw hole 191.

Continuing discussing FIG. 1, in some embodiments, collapsible-locker 101 may comprise means for locking (and unlocking) lid 111 to a main access opening to volume 119 of collapsible-locker 101, when collapsible-locker 101 may be in its substantially expanded (un-folded) configuration. In some embodiments, lid 111 may comprise at least one latch-lock 181. In some embodiments, lid 111 may comprise two latch-locks 181. In some embodiments, located proximate (e.g., within four inches) to each front corner of lid 111 may be a latch-lock 181. In some embodiments, latch-lock(s) 181 may be located under a front lid-overhang 113 of lid 111. In some embodiments, a given latch-lock 181, as its name suggest, may be a lock operating via a latch, wherein this latch may be configured to removably grab onto a given latch-lock-receiver 183. In some embodiments, a given latch-lock-receiver 183 may be region of a bar (i.e., a short elongate-member) and/or a portion of a ring structure. In some embodiments, latch-lock-receiver 183 may be a receiving anchor structure of collapsible-locker 101, configured to removably receive the latch of a given latch-lock 181. In some embodiments, a given latch-lock-receiver 183 may be fixed and/or static with respect to collapsible-locker 101. In some embodiments, a given latch-lock 181 may be electrically activated for locking and/or for unlocking. In some embodiments, a given latch-lock 181 may comprise one or more latches, springs, solenoids, magnets, motors, drivers, combinations thereof, and/or the like.

In some embodiments, when collapsible-locker 101 may be in its substantially expanded (unfolded) configuration (e.g., FIG. 1 and FIG. 3), if lid 111 is pushed (e.g., by a person's hand) onto the main access opening to volume 119 of collapsible-locker 101; latch-lock(s) 181 may automatically lock onto (i.e., audibly click and latch onto) its respective latch-lock-receiver 183, resulting in lid 111 being locked onto front-wall 103.

In some embodiments, a given latch-lock 181 may be operatively connected to (e.g., wired) to various electronics of collapsible-locker 101, such as, but not limited to, Processors 1501, I/O Means 1507, power-supply 1509, batteries, battery pack, bus, switches, circuits, combinations thereof, and/or the like.

Continuing discussing FIG. 1, in some embodiments, for each latch-lock 181, front-wall 103 may comprise a given latch-lock-receiver 183. In some embodiments, a given latch-lock-receiver 183 may be located at (or proximate, i.e., within one inch) to a top of front-wall 103.

Continuing discussing FIG. 1, in some embodiments, collapsible-locker 101 may also comprise means for locking (and unlocking) lid 111 to front-wall 103 of collapsible-locker 101, when collapsible-locker 101 may be in its substantially collapsed (folded) configuration. In some embodiments, for each latch-lock 181, front-wall 103 may comprise a latch-lock-receiver 185. In some embodiments, a given latch-lock-receiver 185 may be located on an exterior surface of front-wall 103, closer to a bottom of front-wall 103 than to the top of front-wall 103. In some embodiments, a given latch-lock-receiver 185 may be region of a bar (i.e., a short elongate-member) and/or a portion of a ring structure. In some embodiments, latch-lock-receiver 185 may be a receiving anchor structure of collapsible-locker 101, configured to removably receive the latch of a given latch-lock 181. In some embodiments, a given latch-lock-receiver 185 may be fixed and/or static with respect to collapsible-locker 101.

In some embodiments, latch-lock-receiver(s) 183 and/or latch-lock-receiver(s) 185 may be located on side-wall(s) 105.

FIG. 2 shows a front view of collapsible-locker 101 in the same configuration as shown in FIG. 1.

FIG. 3 shows a left, front, and top perspective view of a collapsible-locker 101 in its fully expanded configuration with its lid 111 closed. In FIG. 3 (and in FIG. 4) at least some top/upper portions of lid 111 may be shown. In some embodiments, at least one sterilizer 175 may be shown on top/upper portions of lid 111. In some embodiments, sterilizer 175 may be a region of lid 111 that may be both self-sterilizing and when not being sterilized, intended to be engaged by a human hand, such as for pushing lid 111 down to close. In some embodiments, a person may push down on sterilizer 175 to close and lock lid 111 (e.g., lock lid 111 to front-wall 103); thereafter, sterilizer 175 may be electrically activated to sterilize an exterior surface of sterilizer 175.

Continuing discussing FIG. 3, in some embodiments, an exterior surface of collapsible-locker 101 may comprise at least one light/sensor 195. In some embodiments, reference numeral 195 may refer to a light, a light source, a sensor, a hole, a screw hole, a hole for removably receiving an injection molding artefact, an injection molding artefact, combinations thereof, and/or the like. In some embodiments, light/sensor 195 may be a member of I/O Means 1507. In some embodiments, light/sensor 195 may be one or more of: a light source, a light bulb, a light tube, a light emitting diode (LED) (in strip format in some embodiments), incandescent light bulb, fluorescent light tube/bulb, cathode tube/bulb, combinations thereof, and/or the like. In some embodiments, light/sensor 195 may be configured and/or oriented to emit at least some visual light exteriorly of collapsible-locker 101. In some embodiments, light emitted by light/sensor 195 may operate as a night light. In some embodiments, light emitted by light/sensor 195 may help to define and/or indicate a presence of collapsible-locker 101. In some embodiments, light emitted by light/sensor 195 may help to define and/or indicate a footprint of collapsible-locker 101. In some embodiments, light emitted by light/sensor 195 may help to avoid unintended collisions with collapsible-locker 101. In some embodiments, light/sensor 195 may be configured and/or oriented to emit at least some visual light for a purpose of indicating status, warning, alarms, combinations thereof, and/or the like. In some embodiments, light/sensor 195 may emit light to indicate an operational status of sterilizer(s) 171/175, such as, but not limited to, whether or not sterilizer(s) 171/175 may be active, on, off, in need of bulb/tube/light changing, combinations thereof, and/or the like.

Continuing discussing FIG. 3, in some embodiments, light/sensor 195 may be one or more of: a sensor, a thermometer, a thermocouple, a temperature probe, a light meter, a motion detector, radar, sonar, a laser, an accelerometer, a hygrometer, a moisture detector, a water detector, a smoke detector, a particulate detector, combinations thereof, and/or the like. In some embodiments, light/sensor 195 may be configured and/or oriented to sense and/or detect at least one condition that is exterior of collapsible-locker 101.

Continuing discussing FIG. 3, in some embodiments, light/sensor 195 may be located on an exterior surface of collapsible-locker 101. In some embodiments, an exterior surface of collapsible-locker 101 may comprise at least one opening configured to provide light/sensor 195 access to an exterior of collapsible-locker 101.

Continuing discussing FIG. 3, in some embodiments, an exterior of lid 111 may comprise at least one light/sensor 195. In some embodiments, an upper surface of lid 111 may comprise at least one light/sensor 195. In some embodiments, an exterior of lid 111 may comprise at least one opening for light/sensor 195. In some embodiments, an upper surface of lid 111 may comprise at least one opening for light/sensor 195.

Continuing discussing FIG. 3, in some embodiments, an exterior of front-wall 103 may comprise at least one light/sensor 195. In some embodiments, an upper surface of front-wall 103 may comprise at least one light/sensor 195. In some embodiments, an exterior of front-wall 103 may comprise at least one opening for light/sensor 195. In some embodiments, an upper surface of front-wall 103 may comprise at least one opening for light/sensor 195.

In some embodiments, an exterior of side-wall 105 may comprise at least one light/sensor 195. In some embodiments, an upper surface of side-wall 105 may comprise at least one light/sensor 195. In some embodiments, an exterior of side-wall 105 may comprise at least one opening for light/sensor 195. In some embodiments, an upper surface of front-wall 103 may comprise at least one opening for light/sensor 195.

In some embodiments, the at least one light/sensor 195 may be attached to: lid 111, front-wall 103, side-wall 105, side-wall-panel-closer-to-rear-wall 107, side-wall-panel-closer-to-front-wall 109, rear-wall 119, floor 121, combinations thereof, and/or the like.

In some embodiments, a plurality of lights/sensors 195 may be operatively connected to themselves. In some embodiments, light(s)/sensor(s) 195 may be operatively connected to (e.g., wired) to various electronics of collapsible-locker 101, such as, but not limited to, Processors 1501, I/O Means 1507, power-supply 1509, batteries, battery pack, bus, switches, circuits, combinations thereof, and/or the like.

Note in some embodiments, at least one of the reference numerals 195 may instead refer to an injection molding artefact 195, such as, but not limited to a hole configured to removably receive an injection molding port.

Continuing discussing FIG. 3, in some embodiments, collapsible-locker 101 may comprise at least one exterior-interface 115. In some embodiments, exterior-interface 115 may be a member of I/O Means 1507. In some embodiments, exterior-interface 115 may be a means for a person proximate to (next to and/or adjacent to) collapsible-locker 101, to interact, engage, control, unlock lid 111, lock lid 111, activate sterilizer(s) 171/175, deactivate sterilizer(s) 171/175, check status, operate, combinations thereof, and/or the like with respect to collapsible-locker 101. In some embodiments, exterior-interface 115 may comprise one or more of: a screen (such as, but not limited to, a computer screen, a monitor screen, and/or a LCD screen), a touchscreen, a keypad, a touch pad, combinations thereof, and/or the like. In some embodiments, exterior-interface 115 may be substantially as shown and/or described in U.S. nonprovisional patent application Ser. No. 16/414,634, and/or in U.S. provisional patent application Ser. No. 62/929,201.

Continuing discussing FIG. 3, in some embodiments, at least some portions of exterior-interface 115 may be back-lit by one or more lights configured to emit UV light for a purpose of sterilizing exterior surfaces of exterior-interface 115.

Continuing discussing FIG. 3, in some embodiments, collapsible-locker 101 may comprise at least one pod-receiver 351 for each pod. In some embodiments, a given pod may be a component that is removable from collapsible-locker 101. In some embodiments, such pods may be shown and/or described in U.S. provisional patent application Ser. No. 62/929,201. In some embodiments, a given pod may comprise one or more of: a handle (configured to take a load of the entire collapsible-locker 101), exterior camera (such as, but not limited to, exterior-camera 2903), exterior sensor, power-supply 1509, battery, rechargeable battery, combinations thereof, and/or the like. In some embodiments, a given pod-receiver 351 may be a docking bay configured to removably receive a given pod, including with electrical connections from that pod to collapsible-locker 101. In some embodiments, a given pod-receiver 351 may be located at and/or proximate to an upper/top and exterior portion of rear-wall 119.

FIG. 4 shows a front view of collapsible-locker 101 in the same configuration (e.g., lid 111 closed) as shown in FIG. 3.

Note, door-attachment-structures (e.g., door-attachment-structure 151, bottom-door-bracket 153, pivoting J-hook 157, sliding J-hook 159, door-attachment-structure 3000, central-rail 3001, end-bracket 3003, insertable-portion 3005, door-thickness-engagement-portion 3007, strap 3021, cam-ratchet 3023, combinations thereof, and/or the like) and the pod may not be shown in FIG. 1 through FIG. 4. However, these structures may be shown and/or described in U.S. nonprovisional patent application Ser. No. 16/414,634, and/or in U.S. provisional patent application Ser. No. 62/929,201. Various door-attachment-structures are shown in U.S. nonprovisional patent application Ser. No. 16/414,634. And various door-attachment-structures are also shown in U.S. provisional patent application Ser. No. 62/929,201. The pod is shown in U.S. provisional patent application Ser. No. 62/929,201.

In some embodiments, exterior surfaces and/or interior surfaces of collapsible-locker 101 may be configured to not deteriorate after repeated exposure to one or more of: UV light/radiation, soaps, surfactants, cleansers, sanitizers, alcohol, bleach, mild oxidizers, weak acids, acetic acid, vinegar, ammonia, combinations thereof, and/or the like.

In some embodiments, exterior surfaces and/or interior surfaces of collapsible-locker 101 may be substantially smooth to facilitate cleaning, sanitizing, sterilization, combinations thereof, and/or the like of the collapsible-locker 101. In some embodiments, exterior surfaces and/or interior surfaces of collapsible-locker 101 may be substantially smooth to facilitate being sprayed down, wiped down, wiped clean, combinations thereof, and/or the like.

In some embodiments, components, parts, surfaces, combinations thereof, and/or the like of collapsible-locker 101 exposed to UV (ultraviolet) emissions from sterilizers 171/175 and/or from the Sun may be resistant to deterioration, decay, becoming brittle, excessive color changes, combinations thereof, and/or the like from exposure to UV emissions.

In some embodiments, main component parts of collapsible-locker 101, such as, but not limited to, at least most of: front-wall 103, rear-wall 119, side-wall(s) 105, side-wall-panel-closer-to-rear-wall 107, side-wall-panel-closer-to-front-wall 109, lid 111, floor 121, pod-receiver 351, exterior housing of the pod, combinations thereof, and/or the like may be substantially constructed of one or more thermoplastics suitable for injection molding. For example, and without limiting the scope of the present invention, at least most of: front-wall 103, rear-wall 119, side-wall(s) 105, side-wall-panel-closer-to-rear-wall 107, side-wall-panel-closer-to-front-wall 109, lid 111, floor 121, pod-receiver 351, exterior housing of the pod, combinations thereof, and/or the like may be substantially constructed of one or more of the following materials: thermoplastic, acrylonitrile-butadiene styrene (ABS), polyvinyl chloride (PVC), polycarbonate, acrylic, nylon, polypropylene, polyethylene (e.g., HDPE), combinations thereof, and/or the like.

Note with respect to the materials of construction, it is not desired nor intended to thereby unnecessarily limit the present invention by reason of such disclosure.

In some embodiments, a given collapsible-locker 101 with at least one sterilizer 171/175 may be combined with at least one pair of eyeglasses configured to minimize exposure of UV light to a person's eyes when wearing that at least one pair of eyeglasses. In some embodiments, a system for sterilizing at least some portions of a collapsible-locker 101 with at least one sterilizer 171/175 may comprise at least one collapsible-locker 101 and at least one pair of eyeglasses configured to minimize exposure of UV light to a person's eyes when wearing that at least one pair of eyeglasses.

Collapsible-lockers with at least one sterilizer have been described. The foregoing description of the various exemplary embodiments of the invention has been presented for the purposes of illustration and disclosure. It is not intended to be exhaustive or to limit the invention to the precise form disclosed. Many modifications and variations are possible in light of the above teaching without departing from the spirit of the invention.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

What is claimed is:

1. A package-receiving-locker for temporary storage of an object, wherein the package-receiving-locker is attachable to a side-hinged-door; wherein the package-receiving-locker comprises:
a collapsible-container that exists in two operational configurations, a substantially collapsed configuration and a substantially expanded configuration, respectively, wherein the collapsible-container comprises at least one wall that is rigid; wherein the collapsible-container further comprises at least one lid that is hingedly attached to the at least one wall; wherein when the collapsible-container is in the substantially collapsed configuration, the at least one lid is lockable with a locking means to prevent unintended deployment of the collapsible-container into the substantially expanded configuration; wherein the collapsible-container further comprises at least one sterilizer, wherein the at least one sterilizer is configured to sterilize at least an exterior portion of the object; and
a door-attachment-structure; wherein at least a portion of the door-attachment-structure is attached to the at least one wall of the collapsible-container; and other different portions of the door-attachment-structure are attached to the side-hinged-door;
wherein when the collapsible-container is in the substantially expanded configuration the collapsible-container has a main interior volume that is substantially void space for the temporary storage of the object.

2. The package-receiving-locker according to claim 1, wherein the at least one sterilizer is at least partially located on an interior surface of the collapsible-container.

3. The package-receiving-locker according to claim 1, wherein the at least one sterilizer is configured to emit at least some electromagnetic energy into the main interior volume.

4. The package-receiving-locker according to claim 1, wherein an interior surface of the collapsible-container comprises at least one opening configured to emit at least some electromagnetic energy from the at least one sterilizer and into the main interior volume.

5. The package-receiving-locker according to claim 1, wherein an underside of the at least one lid comprises at least a portion of the at least one sterilizer.

6. The package-receiving-locker according to claim 1, wherein an underside of the at least one lid comprises at least one opening configured to emit at least some electromagnetic energy from the at least one sterilizer and into the main interior volume.

7. The package-receiving-locker according to claim 1, wherein the at least one sterilizer is attached to one or more of: the at least one lid or the at least one wall.

8. The package-receiving-locker according to claim 7, wherein the at least one wall comprises:
a front-wall, two side-walls, a rear-wall, and a floor.

9. The package-receiving-locker according to claim 1, wherein the at least one sterilizer is operatively connected to at least some electronics of the collapsible-container.

10. The package-receiving-locker according to claim 1, wherein the at least one sterilizer is electrically powered.

11. The package-receiving-locker according to claim 1, wherein the at least one sterilizer is configured to emit electromagnetic energy of a predetermined characteristic.

12. The package-receiving-locker according to claim 1, wherein the at least one sterilizer is configured to emit ionizing radiation.

13. The package-receiving-locker according to claim 1, wherein the at least one sterilizer is configured to emit electromagnetic energy that is configured to harm genetic material.

14. The package-receiving-locker according to claim 1, wherein the at least one sterilizer comprises at least one light source.

15. The package-receiving-locker according to claim 1, wherein the at least one light source is configured to emit light in the ultraviolet spectrum.

16. The package-receiving-locker according to claim 1, wherein the collapsible-container further comprises interior surfaces that are substantially reflective and configured to reflect at least some light emitted from the at least one sterilizer.

17. The package-receiving-locker according to claim 1, wherein the at least on lid comprises an interior surface that is substantially reflective and configured to reflect at least some light emitted from the at least one sterilizer.

18. The package-receiving-locker according to claim 1, wherein the at least on wall comprises an interior surface that is substantially reflective and configured to reflect at least some light emitted from the at least one sterilizer.

19. The package-receiving-locker according to claim 1, wherein the collapsible-container comprises at least one indicator light on an exterior of the collapsible-container that is configured to indicate an operational status of the at least one sterilizer.

20. The package-receiving-locker according to claim 1, wherein interior surfaces of the collapsible-container that surround the main interior volume are substantially constructed from at least one material that is resistant to deterioration from electromagnetic radiation emitted by the at least one sterilizer.
